(19) United States
(12) Patent Application Publication
(10) Pub. No.: US 2007/0166387 A1
(43) Pub. Date: Jul. 19, 2007

(54) Hemostatic compositions, assemblies, systems, and methods employing particulate hemostatic agents formed from chitosan and including a polymer mesh material of poly-4-hydroxy butyrate

(75) Inventors: Ajay Ahuja, Needham, MA (US); David P. Martin, Arlington, MA (US); Simon J. McCarthy, Portland, OR (US)

Correspondence Address: RYAN KROMHOLZ & MANION, S.C., POST OFFICE BOX 26618, MILWAUKEE, WI, 53226, US

(73) Assignees: HemCon, Inc.; Tepha Inc.

(21) Appl. No.: 11/485,857

(22) Filed: Jul. 13, 2006

Related U.S. Application Data
(60) Provisional application No. 60/698,734, filed on Jul. 13, 2005.

(51) Int. Cl. A61K 9/14

(52) U.S. Cl. 424/489

(57) ABSTRACT

A granule or particle made of a chitosan material either carries within it a polymer mesh material of poly-4-hydroxy butyrate, or has interspersed with it a polymer mesh material of poly-4-hydroxy butyrate. The granule or particle can be carried within a polymer mesh socklet made of a material consisting essentially of poly-4-hydroxy butyrate. The granule or particle can be used to treat intracavity bleeding.

### RELATED APPLICATIONS

This application claims the benefit of U.S. Provisional Application Ser. No. 60/698,734, filed Jul. 13, 2005, and entitled “Hemostatic Compositions, Assemblies, Systems, and Methods Employing Particulate Hemostatic Agents Formed from Hydrophilic Polymer Foam Such as Chitosan.”

### FIELD OF THE INVENTION

The invention is generally directed to agents applied externally or internally on a site of tissue injury or tissue trauma to ameliorate bleeding, fluid seepage or weeping, or other forms of fluid loss.

### BACKGROUND OF THE INVENTION

Hemorrhage is the leading cause of death from battlefield trauma and the second leading cause of death after trauma in the civilian community. Non-compressible hemorrhage (hemorrhage not readily accessible to direct pressure, such as intracavity bleeding) contributes to the majority of early trauma deaths. Apart from proposals to apply a liquid hemostatic foam and recombinant factor VIIa to the non-compressible bleeding sites, very little has been done to address this problem. There is a critical need to provide more effective treatment options to the combat medic for controlling severe internal hemorrhage such as intracavity bleeding.

Control of intracavity bleeding is complicated by many factors, chief among which are: lack of accessibility by conventional methods of hemostatic control such as application of pressure and topical dressings; difficulty in assessing the extent and location of injury; bowel perforation, and interferences caused by blood flow and pooling of bodily fluids.

### SUMMARY OF THE INVENTION

The invention provides a chitosan hemostatic agent matrix in the form of a granule or particle that carries within it a polymer mesh material formed from poly-4-hydroxy butyrate (TephaFLEX™ Material manufactured by Tepha Inc.).

The invention also provides a chitosan hemostatic agent matrix as just described that can be applied within a polymer mesh socklet formed from poly-4-hydroxy butyrate (TephaFLEX™ Material manufactured by Tepha Inc.).

The improved hemostatic agents as just described can be used to stanch, seal, or stabilize a site of noncompressible hemorrhage, e.g., at a site of intracavity bleeding. The invention provides rapid delivery of a safe and effective hemostatic agent to a general site of bleeding; enhanced promotion of strong clot formation at the site of bleeding; and ability (if necessary) to apply tamponade over the field of injury. The invention also provides an enhanced rate of wound healing with reduced fibrotic adhesion and reduced opportunity for wound infection. The invention therefore addresses many of the significant issues related to current difficulties in controlling intracavitary hemorrhage and recovery from this type of injury.

Other features and advantages of the invention shall be apparent based upon the accompanying description, drawings, and listing of key technical features.

### DESCRIPTION OF THE DRAWINGS

FIG. 1A is a schematic anatomic view of an intracavity site of noncompressible hemorrhage, into which a hemostatic agent has been applied to stanch, seal, or stabilize the site.

FIG. 1B is an enlarged view of the hemostatic agent shown in FIG. 1A, showing the granules or particles that comprise the agent.

FIG. 2 is a further enlarged view of the granules or particles shown in FIG. 1B showing strips of a polymer mesh material formed from poly-4-hydroxy butyrate (TephaFLEX™ Material manufactured by Tepha Inc.) that have been added to the granules or particles.

FIG. 3 is a schematic flow chart view of a process of manufacturing the granules or particles shown in FIG. 2 from a chitosan material.

FIG. 4 shows a step in the manufacturing process shown in FIG. 3, in which strips of the polymer mesh material formed from poly-4-hydroxy butyrate (TephaFLEX™ Material manufactured by Tepha Inc.) are added to the granules or particles.

FIG. 5 shows a composite hemostatic agent comprising hemostatic granules or particles mixed with strips of polymer mesh material formed from poly-4-hydroxy butyrate (TephaFLEX™ Material manufactured by Tepha Inc.).

FIG. 6 shows a bolus of the granules or particles shown in FIG. 2 contained for delivery in a socklet of polymer mesh material formed from poly-4-hydroxy butyrate (TephaFLEX™ Material manufactured by Tepha Inc.).

FIG. 7 shows one way of delivering the bolus of the granules or particles shown in FIG. 6 in the socklet of polymer mesh material to an injury site.

FIGS. 8A and 8B show a way of delivering a bolus of the granules or particles shown in FIG. 2 into a releasable polymer mesh socklet formed from poly-4-hydroxy butyrate (TephaFLEX™ Material manufactured by Tepha Inc.) at an injury site.

FIG. 9 is an alternative way of delivering a bolus of the granules or particles shown in FIG. 2 to an injury site without use of a containment socklet or the like.

### DETAILED DESCRIPTION

Although the disclosure hereof is detailed and exact to enable those skilled in the art to practice the invention, the physical embodiments herein disclosed merely exemplify the invention, which may be embodied in other specific structure. While the preferred embodiment has been described, the details may be changed without departing from the invention, which is defined by the claims.

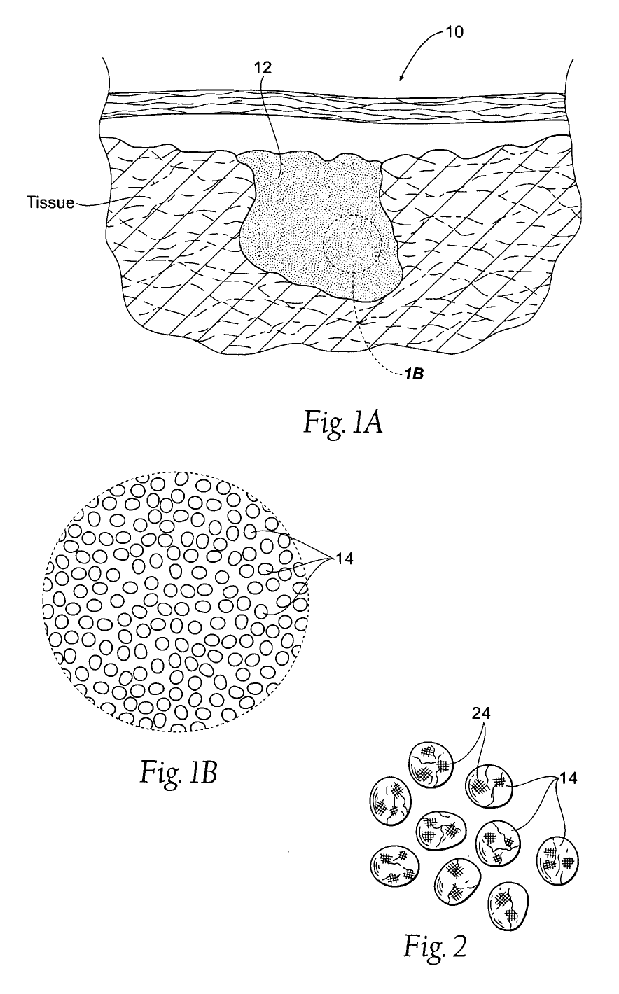

FIG. 1A shows a site 10 of an intracavity abdominal injury, where severe internal bleeding will occur if steps are not taken to stanch, seal, or stabilize the site. The site 10 is the location of a noncompressible hemorrhage, meaning that the hemorrhage is not readily accessible to direct pressure.

As shown in FIGS. 1A and 1B, a hemostatic agent 12 that embodies the features of the invention has been applied to stanch, seal, or stabilize the site 10 without the application of direct pressure or compression. The agent 12 takes the form of discrete particles 14 of a biodegradable hydrophilic polymer (best shown in FIG. 1B and FIG. 2).

The polymer of which the particles 14 are formed has been selected to include a biocompatible material that reacts in the presence of blood, body fluid, or moisture to become a strong adhesive or glue. Desirably, the polymer from which the particles 14 are formed also desirably possess other beneficial attributes, for example, anti-bacterial and/or anti-microbial anti-viral characteristics, and/or characteristics that accelerate or otherwise enhance the body's defensive reaction to injury. The polymer material comprising the particles 14 has desirably been densified or otherwise treated to make the particles 14 resistant to dispersal away from the site 10 by flowing blood and/or other dynamic conditions affecting the site 10.

The agent 12 thereby serves to stanch, seal, and/or stabilize the site 10 against bleeding, fluid seepage or weeping, or other forms of fluid loss. The agent 12 also desirably forms an anti-bacterial and/or anti-microbial and/or anti-viral protective barrier at or surrounding the tissue treatment site 10. The agent 12 can applied as temporary intervention to stanch, seal, and/or stabilize the site 10 on an acute basis. The agent 12 can also be augmented, as will be described later, to make possible more permanent internal use.

The particles 14 shown in FIG. 2 comprise a chitosan material, most preferably poly [β-(1→4)-2-amino-2-deoxy-D-glucopyranose. The chitosan selected for the particles 14 preferably has a weight average molecular weight of at least about 100 kDa, and more preferably, of at least about 150 kDa. Most preferably, the chitosan has a weight average molecular weight of at least about 300 kDa.

The chitosan can be manufactured in the manner described in U.S. patent application Ser. No. 11/020,365 filed on Dec. 23, 2004, entitled “Tissue Dressing Assemblies, Systems, and Methods Formed From Hydrophilic Polymer Sponge Structures Such as Chitosan”; U.S. patent application Ser. No. 10/743,052, filed on Dec. 23, 2004, entitled “Wound Dressing and Method of Controlling Severe Life-Threatening Bleeding”; U.S. patent application Ser. No. 10/480,827, filed on Dec. 15, 2003, entitled “Wound Dressing and Method of Controlling Severe Life-Threatening Bleeding,” which was a national stage filing under 37 C.F.R. § 371 of International Application No. PCT/US02/18757, filed on Jun. 14, 2002, which are each incorporated herein by reference.

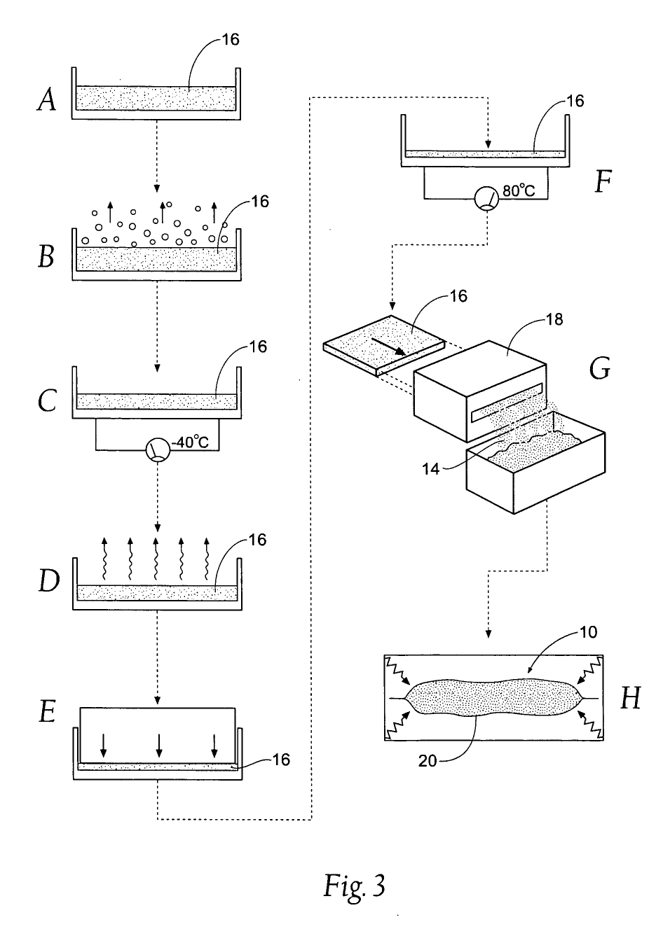

Generally speaking the chitosan particles 14 are formed by the preparation of a chitosan solution by addition of water to solid chitosan flake or powder at 25° C. (FIG. 3, Step A), the solid being dispersed in the liquid by agitation, stirring or shaking. On dispersion of the chitosan in the liquid, the acid component is added and mixed through the dispersion to cause dissolution of the chitosan solid. The chitosan biomaterial 16 is desirably degassed of general atmospheric gases (FIG. 3, Step B). The structure or form producing steps for the chitosan material 16 are typically carried out from solution and can be accomplished employing techniques such as freezing (to cause phase separation) (FIG. 3, Step C). In the case of freezing, where two or more distinct phases are formed by freezing (typically water freezing into ice with differentiation of the chitosan biomaterial into a separate solid phase), another step is required to remove the frozen solvent (typically ice), and hence produce the chitosan matrix 16 without disturbing the frozen structure. This may be accomplished by a freeze-drying and/or a freeze substitution step (FIG. 3, Step D).

The chitosan material 16 comprise an “uncompressed” chitosan acetate matrix of density less than 0.035 g/cm³ that has been formed by freezing and lyophilizing a chitosan acetate solution, which is then densified by compression (FIG. 3, Step E) to a density of from 0.6 to 0.5 g/cm³, with a most preferred density of about 0.25 to 0.5 g/cm³. This chitosan matrix can also be characterized as a compressed, hydrophilic sponge structure. The densified chitosan matrix 16 exhibits all of the above-described characteristics deemed to be desirable. It also possesses certain structural and mechanical benefits that lend robustness and longevity to the matrix during use, as will be described in greater detail later.

The densified chitosan biomaterial 16 is next preferably preconditioned by heating chitosan matrix 16 in an oven to a temperature of preferably up to about 75° C., more preferably to a temperature of up to about 80° C., and most preferably to a temperature of preferably up to about 85° C. (FIG. 3, Step F).

After formation in the manner just described, the sponge structure is granulated, e.g., by a mechanical process, to a desired particle diameter, e.g., at or near 0.9 mm. Simple mechanical granulation of the chitosan matrix 16 through a suitable mechanical device 18 (as shown in FIG. 3, Step G) can be used to prepare chitosan sponge particles 14 of close to 0.9 mm in diameter. Other granulation methodologies can be used. For example, off the shelf stainless steel grinding/granulating laboratory/food processing equipment can be used. More robust, purpose designed, and more process-controlled systems can also be used. Granulation of the chitosan matrix 16 can be conducted under ambient temperature or liquid nitrogen temperature conditions.

Preferably, a well defined particle size distribution of particle granulate 14 is prepared. The particle size distribution can be characterized using, e.g., Leica ZP6 APO stereomicroscope and Image Analysis MC software. The granulated particles are sterilized (FIG. 3, Step H), for example, by irradiation, such as by gamma irradiation.

The chitosan matrix from which the particles 14 are formed presents a robust, permeable, high specific surface area, positively charged surface. The positively charged surface creates a highly reactive surface for red blood cell and platelet interaction. Red blood cell membranes are negatively charged, and they are attracted to the chitosan matrix. The cellular membranes fuse to chitosan matrix upon contact. A clot can be formed very quickly, circumventing immediate need for clotting proteins that are normally required for hemostasis. For this reason, the chitosan matrix is effective for both normal as well as anti-coagulated individuals, and as well as persons having a coagulation disorder like hemophilia. The chitosan matrix also binds bacteria, endotoxins, and microbes, and can kill bacteria, microbes, and/or viral agents on contact. Furthermore, chitosan is biodegradable within the body and is broken down into glucosamine, a benign substance.

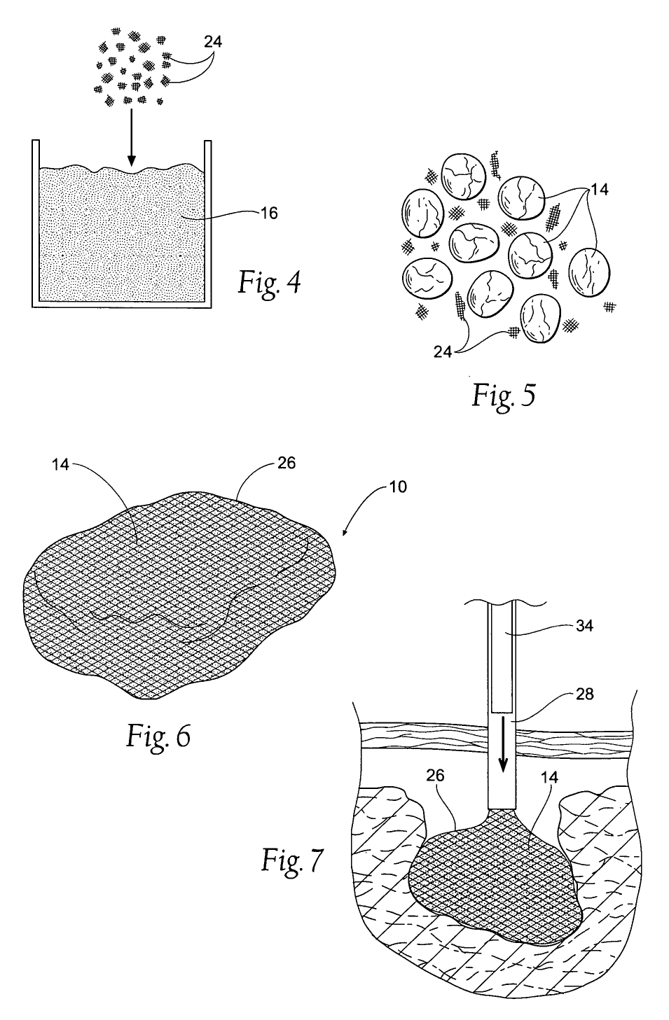

The interior of the particles 14 can be reinforced by the inclusion of small strips or pieces of a bioresorbable polymer mesh material 24 (as shown in FIG. 2) formed from poly-4-hydroxy butyrate (TephaFLEX™ Material manufactured by Tepha Inc.). These strips of mesh material 24 can be added to the viscous chitosan solution 16 immediately before the freezing step (as FIG. 4 shows). Alternatively (as FIG. 5 shows), loose small strips or pieces of the bioresorbable poly-4-hydroxy butyrate (TephaFLEX™ Material manufactured by Tepha Inc.) mesh material 24 can be added after granulation and prior to pouching and sterilization. In this arrangement, the strips or pieces of the mesh material 24 reside between the individual particles 14 contained within the pouch 22 (as shown in FIG. 5).

The presence of the poly-4-hydroxy butyrate (TephaFLEX™ Material manufactured by Tepha Inc.) mesh material 24 enhances hemostasis by overall reinforcement of the complex composite of chitosan granule particle 14, blood, and the mesh material 24.

The poly-4-hydroxy butyrate (TephaFLEX™ Material manufactured by Tepha Inc.) mesh material is a biosynthetic absorbable polyester produced through a fermentation process rather than by chemical synthesis. It can generally be described as a strong, pliable thermoplastic with a tensile strength of 50 MPa, tensile modulus of 70 MPa, elongation to break of ˜1000%, and hardness (Shore D) of 52.8. Upon orientation the tensile strength increases approximately 10-fold (to a value about 25% higher than commercial absorbable monofilament suture materials such as PDSII™).

Despite its biosynthesis route, the structure of the polyester is very simple, and closely resembles the structures of other existing synthetic absorbable biomaterials used in medical applications. The polymer belongs to a larger class of materials called polyhydroxyalkanoates (PHAs) that are produced in nature by numerous microorganisms. In nature these polyesters are produced as storage granules inside cells, and serve to regulate energy metabolism. They are also of commercial interest because of their thermoplastic properties, and relative ease of production. Tepha, Inc. produces the TephaFLEX™ biomaterial for medical applications using a proprietary transgenic fermentation process specifically engineered to produce this homopolymer. The TephaFLEX™ biomaterial production process utilizes a genetically engineered Escherichia coli K12 microorganism that incorporates new biosynthetic pathways to produce the polymer. The polymer accumulates inside the fermented cells during fermentation as distinct granules, and can then be extracted at the end of the process in a highly pure form. The biomaterial has passed tests for the following: cytotoxicity; sensitization; irritation and intracutaneous reactivity; hemocompatibility; endotoxin; implantation (subcutaneous and intramuscular); and USP Class VI. In vivo, the TephaFLEX™ biomaterial is hydrolyzed to 4-hydroxybutyrate, a natural human metabolite, present normally in the brain, heart, lung, liver, kidney, and muscle. This metabolite has a half-life of just 35 minutes, and is rapidly eliminated from the body (via the Krebs cycle) primarily as expired carbon dioxide.

Being thermoplastic, the TephaFLEX™ biopolymer can be converted into a wide variety of fabricated forms using traditional plastics processing technologies, such as injection molding or extrusion. Melt extruded fibers made from this novel absorbable polymer are at least 30% stronger, significantly more flexible and retain their strength longer than the commercially available absorbable monofilament suture materials. These properties make the TephaFLEX™ biopolymer an excellent choice for construction of a hemostatic dressing for controlling intracavity hemorrhage.

The TephaFLEX™ biomaterial can be processed into fibers and fabrics suitable for use as an absorbable sponge.

To provide for enhanced local delivery and potentially some pressure compaction (tamponade) of the encased granulate against the wound, the chitosan granulate particles 14 can be desirable housed for delivery within an open mesh socklet or bag 26 (see FIG. 6) made from a TephaFLEX biomaterial above described.

The mesh of the socklet 26 is sufficiently open to allow for the chitosan granulate particles 14 to protrude out of the socklet 26, but not so open that granulate particles 14 could be flushed away by flowing blood through the mesh. The socklet 26 supports the chitosan granulate particles 14 during and after delivery and allows a more directed application of a bolus of the granulate particles 14. The mesh socklet 26 should be sufficiently open to allow protrusion of chitosan particles 14 at the outer surface of the bolus from its outside surface without loss of individual chitosan granule particles 14. The mechanical properties of the mesh socklet 26 are sufficient to allow local application of pressure over its surface without tearing or breaking.

The tamponade of a socklet 26 filled with the particles 14 can be applied, e.g., through a cannula 28 (see FIG. 7) by use of tamp 34 to advance the socklet 26 through the cannula 28 to the injury site 10. Multiple socklets 26 can be delivered in sequence through the cannula 28, if required. Alternatively, a caregiver can manually insert one or more of the socklets 26 into the treatment site 10 through a surface incision.

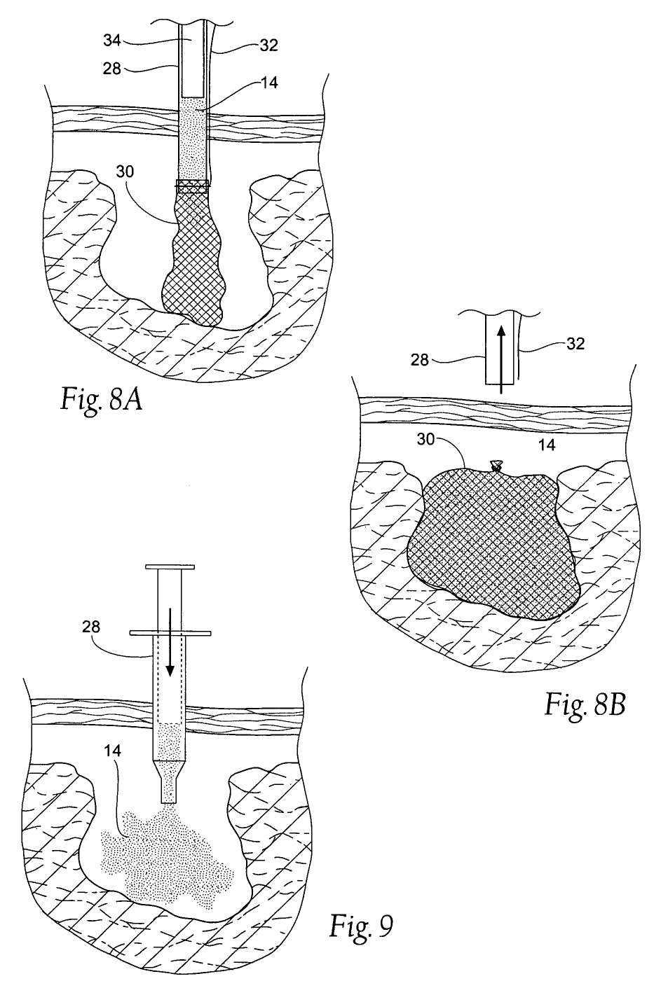

Alternatively, as FIGS. 8A and 8B show, a mesh socklet 30 can be releasably attached to the end of a cannula 28, e.g., by a releasable suture 32. The cannula 28 guides the empty socklet 30 into the injury site 10. In this arrangement, individual particles 14 (i.e., not confined during delivery within a mesh socklet 26 as shown in FIG. 6) can be urged through the cannula 28, using, e.g., a tamp, to fill the socklet 30 within the injury site. Upon filling the socklet 30 with particles 14, the suture 32 can be pulled to release the cannula 28, leaving the particle filled socklet 30 behind in the injury site 10, as FIG. 8B shows.

Alternatively, as FIG. 9 shows, individual particles. 14 can be delivered to the injury site 10 through a syringe 36. In this arrangement, means for targeting of the particles 14 at the injury site 10 and protection against disbursement of the particles 14 away from the injury site 10 due to blood flow may be required, using the already described confinement devices and techniques. It is believed that permanent internal use will require the use of a socklet or equivalent confinement technique.

Therefore, it should be apparent that above-described embodiments of this invention are merely descriptive of its principles and are not to be limited. The scope of this invention instead shall be determined from the scope of the following claims, including their equivalents.

We claim:

1. A hemostatic agent comprising a granule or particle made of a chitosan material and a polymer mesh material consisting essentially of poly-4-hydroxy butyrate carried within the granule or particle.

2. An assembly comprising a hemostatic agent that takes the form of a granule or particle made of a chitosan material and strips of pieces of a polymer mesh material consisting essentially of poly-4-hydroxy butyrate interspersed with the hemostatic agent.

3. An assembly comprising a polymer mesh socklet made of a material consisting essentially of poly-4-hydroxy butyrate and a hemostatic agent that takes the form of a granule or particle made of chitosan material carried within the socklet.

4. Methods of treat intracavity bleeding using the materials defined in claims 1 or 2 or 3.
